(19) United States
(12) Patent Application Publication
(10) Pub. No.: US 2012/0238596 A1
(43) Pub. Date: Sep. 20, 2012

(54) FORMULATIONS COMPRISING PALONOSETRON

(75) Inventors: Chandrashekhar Kocherlakota, Secunderabad (IN); Sreedhar Bandari, Karimnagar (IN); Nagaraju Banda, Hyderabad (IN); Kranthi Kumar Hinge, Warangal (IN); Ramachandra Reddy Yeluri, Prakasam (IN)

(73) Assignees: DR. REDDY'S LABORATORIES, INC., Bridgewater, NJ (US); DR. REDDY'S LABORATORIES LIMITED, Hyderabad (IN)

(21) Appl. No.: 13/483,582

(22) Filed: May 30, 2012

Related U.S. Application Data
(63) Continuation of application No. 12/545,955, filed on Aug. 24, 2009, now abandoned.
(60) Provisional application No. 61/103,975, filed on Oct. 9, 2008.

(30) Foreign Application Priority Data
Aug. 25, 2008 (IN) 2060/CHE/2008

(51) Int. Cl. A61K 31/473; A61P 1/08

(52) U.S. Cl. 514/296

(57) ABSTRACT

Pharmaceutical compositions comprising palonosetron and its pharmaceutically acceptable salts, in the form of a ready-to-use solution or a lyophilized form. Specific embodiments of the invention relate to stable pharmaceutical formulations of palonosetron and its pharmaceutically acceptable salts, wherein the formulation does not comprise a chelating agent or an antioxidant.

Aspects of the present invention relate to pharmaceutical compositions comprising palonosetron, including its pharmaceutically acceptable salts or solvates, in the form of ready-to-use solutions or lyophilized forms and preparations thereof. In specific aspects, the invention includes pharmaceutical compositions with improved stability comprising palonosetron, including its pharmaceutically acceptable salts or solvates, in the form of ready-to-use solutions or lyophilized forms and preparations thereof. Further aspects include processes for preparing such compositions and methods of using such compositions for treating chemotherapy induced emesis and post operative nausea and vomiting in mammals.

Cancer chemotherapy may be associated with a high incidence of nausea and vomiting, particularly when certain agents, such as cisplatin, are used. 5-HT3 receptors are located on the nerve terminals of the vagus in the periphery and centrally in the chemoreceptor trigger zone of the area postrema. It is thought that chemotherapeutic agents produce nausea and vomiting by releasing serotonin from the enterochromaffin cells of the small intestine and that the released serotonin then activates 5-HT3 receptors located on vagal afferents to initiate the vomiting reflex. Postoperative nausea and vomiting is influenced by multiple patient, surgical and anesthesia related factors and is triggered by release of 5-HT in a cascade of neuronal events involving both the central nervous system and the gastrointestinal tract. The 5-HT3 receptor has been demonstrated to selectively participate in the emetic response.

Emesis is a devastating consequence of cytotoxic therapy, radiotherapy, and post-operative environments, and drastically affects the quality of life of people undergoing such treatments. In recent years a class of drugs referred to as 5-HT3 (5-hydroxytryptamine) receptor antagonists has been developed that treat such emesis by antagonizing cerebral functions associated with the 5-HT3 receptor. Drugs within this class include ondansetron, granisetron, alosetron, tropisetron, and dolasetron. These 5-HT3 antagonists are often administered intravenously shortly before chemotherapy or radiotherapy is initiated, and can be administered more than once during a cycle of chemotherapy or radiotherapy. In addition, they are often supplied as tablets or oral elixirs to either supplement an intravenous administration, or to ease home usage of the drug if the patient is self-administering the chemotherapeutic regimen.

Because some chemotherapeutic agents can induce emesis over extended periods of several days even when they are administered only once, it would be desirable to administer an emesis-inhibiting drug such as a 5-HT3 antagonist every day until the risk of emesis has substantially subsided. The present class of 5-HT3 antagonists has not proven especially helpful meeting this need, however, because the 5-HT3 receptor antagonists currently marketed have proven to be less effective in controlling delayed nausea and vomiting than they are at controlling acute emesis.

U.S. Pat. No. 5,202,333 relates to novel compounds which are 5-HT3 receptor antagonists, pharmaceutical compositions containing them and methods for their use and methods for preparing these compounds. These compounds are more potent than most currently used 5-HT3 receptor antagonists, have a surprising half-life of about 40 hours, and are effective to reduce delayed-onset nausea induced by chemotherapeutic agents. In particular, this patent relates to tricyclic 5-HT3 receptor antagonists containing a bridged bicyclic amine substituent, such as palonosetron. This patent discloses an intravenous formulation of palonosetron in an example which contains the following ingredients: palonosetron hydrochloride, dextrose monohydrate, citric acid monohydrate, sodium hydroxide and water for injection.

Palonosetron hydrochloride is a serotonin subtype 3 (5-HT3) receptor antagonist with a strong binding affinity for this receptor. Chemical names for palonosetron hydrochloride include: (3a5)-2-[(S)-1-Azabicyclo [2.2.2] oct-3-yl]-2,3,3a,4,5, 6-hexahydro-1-oxo-1H-benz [de]isoquinoline hydrochloride; and (3aR)-2-[(3S)-1-azabicyclo[2,2,2]oct-3-yl]-2,3,3a,4,5,6-hexahydro-1H-benz[de]isoquinolin-1-one hydrochloride. The empirical formula is C₁₉H₂₄N₂O.HCl, with a molecular weight of 332.87.

Palonosetron hydrochloride is a white to off-white crystalline powder. It is freely soluble in water, soluble in propylene glycol, and slightly soluble in ethanol and 2-propanol. Palonosetron hydrochloride exists as a single isomer and has the following structural formula:

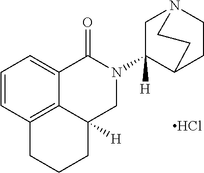

Commercially, palonosetron hydrochloride is available in intravenous injection products sold as ALOXI® in the United States by Eisai Inc. ALOXI® injection is a sterile, clear, colorless, non pyrogenic, isotonic, buffered solution for intravenous administration, available as a 5 mL single use vial or a 1.5 mL single use vial. Each 5 mL vial contains 0.25 mg palonosetron base as the hydrochloride, 207.5 mg mannitol, disodium edetate and citrate buffer in water for intravenous administration. Each 1.5 mL vial contains 0.075 mg palonosetron base as the hydrochloride, 83 mg mannitol, disodium edetate and citrate buffer in water for intravenous administration. The pH of the solution in the 5 mL and 1.5 mL vials is 4.5 to 5.5.

Formulating palonosetron has not proven to be an easy task, typically due to stability issues. Therefore, there remains a need for preparing a palonosetron formulation with improved stability and thereby increased product shelf life.

U.S. Patent Application Publication Nos. 2006/0069114, 2006/0167071, 2006/0167072, and 2006/0167073 describe pharmaceutically stable solutions for reducing emesis or reducing the likelihood of emesis comprising: a) from 0.03 mg/ml to 0.2 mg/ml of palonosetron or a pharmaceutically acceptable salt thereof; b) a chelating agent; and c) a pharmaceutically acceptable carrier; wherein the pharmaceutically acceptable carrier comprises mannitol, at a pH from 4 to 6.

However, there exists a need for an appropriate range of concentrations for both the palonosetron and its pharmaceutically acceptable carriers and an appropriate range of pH that would facilitate making a palonosetron formulation with improved stability.

There also exists a need for formulations of palonosetron, other than ready-to-use solutions for parenteral administration.

### SUMMARY

Aspects of the present invention relate to pharmaceutical compositions comprising palonosetron, including its pharmaceutically acceptable salts or solvates, in the form of ready-to-use solutions or lyophilized forms, and preparations thereof. In specific aspects, the invention includes pharmaceutical compositions with improved stability comprising palonosetron, including its pharmaceutically acceptable salts or solvates, in the form of ready-to-use solutions or lyophilized forms and preparations thereof. Other aspects include processes for preparing such compositions and methods of using such compositions for treating chemotherapy induced emesis and post operative nausea and vomiting in mammals.

An aspect of the present invention provides methods for adjusting the pH and/or excipient concentrations to increase the stability of palonosetron formulations.

Another aspect of the present invention provides simple, rapid and inexpensive manufacturing processes for preparing stable ready-to-use solutions comprising palonosetron.

Another aspect of the present invention provides methods for preparing stable lyophilized formulations of palonosetron.

Another aspect of the invention provides stable formulations comprising palonosetron that allow for long-term storage and also allow terminal sterilization.

Another aspect of the invention provides formulations comprising palonosetron hydrochloride with improved stability for reducing long-term emesis.

Another aspect of the invention provides stable palonosetron formulations for preventing or reducing emesis comprising: a) palonosetron or a pharmaceutically acceptable salt thereof; and b) a pharmaceutically acceptable carrier; wherein the pharmaceutically acceptable carrier comprises a chelating agent, mannitol, pH adjusting agent and preservative.

Another aspect of the invention is to provide stable palonosetron formulations by adjusting the pH of the formulation to less than 4 and more than 6.

Another aspect of the invention provides stable formulations of palonosetron with or without a chelating agent.

### DETAILED DESCRIPTION

In aspects, the present invention relates to pharmaceutical compositions comprising palonosetron, including its pharmaceutically acceptable salts or solvates, in the form of ready-to-use solutions or lyophilized forms, and preparations thereof. Other aspects of the invention include pharmaceutical compositions with improved stability comprising palonosetron, including its pharmaceutically acceptable salts or solvates, in the form of ready-to-use solutions or lyophilized forms, and preparations thereof. Further aspects include processes for preparing such compositions and methods of using such compositions for treating chemotherapy induced emesis and post operative nausea and vomiting in mammals.

Injectable formulations are typically formulated as aqueous solutions in which water is the primary excipient. Injectable formulations can be prepared in conventional forms, either as liquid solutions or suspensions, solid forms suitable for solubilization or suspension in liquid prior to injection, or as emulsions. Sterile injectable formulations can be prepared according to techniques known in the art using suitable carriers, dispersing or wetting agents, and suspending agents. The injectable formulations may be sterile injectable solutions or suspensions in a nontoxic, parenterally acceptable diluent or solvent. Among the acceptable vehicles and solvents that may be employed are water, Ringer's solution and isotonic sodium chloride solution. In addition, sterile fixed oils, fatty esters, or polyols are conventionally employed as solvents or suspending media.

The formulations of the present invention are particularly suited for use in parenteral administration, but it will be understood that the solutions may have alternative uses. For example, they may be used as intermediates in the preparation of other pharmaceutical dosage forms. Similarly, they may have other routes of administration including intranasal or inhalation. Injectable formulations may take any route including intramuscular, intravenous or subcutaneous.

Also provided are processes for the preparation of injectable pharmaceutical formulations. Certain processes include lyophilizing or freeze-drying aqueous solutions, such as an alkaline or acidic aqueous solution, which comprises palonosetron and pharmaceutically acceptable excipients.

The injectable pharmaceutical formulations of the present invention may optionally include one or more pharmaceutically acceptable excipients. The pharmaceutically acceptable excipients may include one or more: antibacterial preservatives, including one or more of phenylmercuric nitrate, thiomersal, benzalkonium chloride, benzethonium chloride, phenol, cresol and chlorobutanol; antioxidants including one or more of ascorbic acid, sodium sulfite, sodium bisulfite and sodium metabisulfite; chelating agents such as ethylenediamine tetraacetic acid (EDTA) and its derivatives; buffers including one or more of acetate, citrate, tartarate, phosphate, benzoate and bicarbonate; tonicity contributors including one or more of sodium chloride, potassium chloride, dextrose, mannitol, sorbitol and lactose; and alkaline substances including one or more of sodium hydroxide, potassium hydroxide, sodium carbonate and meglumine.

Antioxidants or chelating agents may be employed to protect the active agent from oxidative degradation, particularly under the rigorous conditions of thermal sterilization. The amount of antioxidant employed can be determined using routine experimentation. As an alternative to the use of antioxidant compounds, the antioxidant effect can be achieved by displacing oxygen (air) from contact with the solution of active agent. This is usually carried out by purging a container holding the solution with an inert gas, e.g., nitrogen.

The term “pharmaceutically acceptable” describes an ingredient that is useful in preparing a pharmaceutical composition and is generally safe, non-toxic and neither biologically nor otherwise undesirable, and includes those acceptable for veterinary use as well as human pharmaceutical use.

The term “stable pharmaceutical formulation” refers to any preparation of palonosetron having sufficient stability to allow storage at a convenient temperature, such as between about 0° C. and about 60° C., for a commercially reasonable period of time, such as at least about one week, at least about one month, at least about three months, at least about six months, at least about one year, or at least about 2 years.

The addition of a tonicity contributor, such as any of the sugars like dextrose, sucrose, mannose and the like, can improve the stability of palonosetron formulations, with or without a chelating agent.

Therefore, in an embodiment, the invention provides a pharmaceutically stable solution for preventing or reducing emesis comprising: a) palonosetron or a pharmaceutically acceptable salt thereof; and b) a pharmaceutically acceptable carrier; wherein the pharmaceutically acceptable carrier comprises a tonicity contributor and may, or may not, comprise a chelating agent.

Similarly, in another embodiment the invention provides a method of formulating a pharmaceutically stable solution of palonosetron comprising admixing: a) palonosetron or a pharmaceutically acceptable salt thereof; and b) a pharmaceutically acceptable carrier; wherein the pharmaceutically acceptable carrier comprises a chelating agent and mannitol. The chelating agent is a material such as EDTA, and, in various embodiments the chelating agent is present in a concentration of from about 0.065 to about 1 mg/mL.

In embodiments, the formulations are substantially free of chelating agents, antioxidants, or both.

In various embodiments, the tonicity contributor is present in a concentration of from about 10 mg/mL to about 80 mg/mL.

In an another embodiment the invention provides a pharmaceutically stable aqueous solution for preventing or reducing emesis comprising: from about 0.01 to about 5 mg/ml of palonosetron or a pharmaceutically acceptable salt thereof; from about 10 to about 100 millimoles/mL of citrate buffer; and from about 0.005 to about 1 mg/mL of EDTA.

In another embodiment the invention provides a method of formulating a pharmaceutically acceptable solution with improved stability of palonosetron comprising admixing from about 0.01 mg/mL to about 7.5 mg/mL of palonosetron, or a pharmaceutically acceptable salt thereof, with a pharmaceutically acceptable carrier.

The formulations of the present invention can be prepared according to conventional techniques and the pH of the final preparation is less than about 8, and frequently will be less than about 4 or more than about 6, but a pH between about 4 and about 6 also can be achieved. If necessary the pH of the final formulation is adjusted to the desired value by adding an acid or base, as appropriate.

In an embodiment, a stable palonosetron formulation comprises: a) palonosetron or a pharmaceutically acceptable salt thereof; and b) a pharmaceutically acceptable carrier without a chelating agent; at a pH from about 4 to about 6.

The stable palonosetron formulations of this invention can be in the form of a ready-to-use dosage form or can be in the form of a lyophilized preparation, which can be reconstituted by mixing with a diluent before administration.

The stable palonosetron formulations also can be subjected to terminal sterilization processes in the manufacturing process, and the stable palonosetron solutions can be lyophilized and filled into containers.

In embodiments, the invention provides methods of filling a container which contains a solution or lyophilized powder of palonosetron or a pharmaceutically acceptable salt thereof comprising: a) providing one or more sterile open containers (e.g., 5 mL vials); b) filling the containers with a solution or lyophilized powder of palonosetron in a non-aseptic environment; c) sealing the filled containers; and d) sterilizing the sealed, filled containers.

Formulations are prepared using palonosetron particles having mean particle sizes of about 1 μm to about 200 μm, about 3 μm to about 100 μm, or about 5 μm to about 50 μm.

As used herein, the term “mean particle size” refers to a distribution of particles wherein about 50 volume percent of all particles measured have particle sizes less than the defined mean particle size value, and about 50 volume percent of all measurable particles measured have particle sizes greater than the defined mean particle size value; this can be denoted by the term “D₅₀.” Similarly, a particle size distribution parameter where 90 volume percent of the particles have sizes less than a specified size is referred to as “D₉₀” and a distribution parameter where 10 volume percent of particles have sizes less than a specified size is referred to as “D₁₀.” A desired particle size range material can be obtained directly from a synthesis process or any known particle size reduction processes can be used, such as but not limited to sifting, milling, micronization, fluid energy milling, ball milling, and the like. Methods for determining D₁₀, D₅₀ and D₉₀ include laser light diffraction, such as using equipment from Malvern Instruments Ltd. (Malvern, Worcestershire, United Kingdom), as well as other techniques known to those having skill in the art.

Vials are small, usually glass, containers that are sealed with a suitable stopper and seal, and other suitable primary containers may be used, for instance, but not limited to, pre-filled syringes. Vials also are sealed containers of medication that are used one time only, and includes breakable and non-breakable closed glass containers, breakable plastic containers, miniature screw-top jars, and any other type of container of a size capable of holding only one unit dose of palonosetron (typically about 5 mL).

The invention includes use of packaging materials such as containers and closures of high-density polyethylene (HDPE), low-density polyethylene (LDPE) and or polypropylene and/or glass, glassine foil, polyvinyl chloride, polyvinylidene dichloride, etc.

The pharmaceutical dosage forms of the present invention are intended for parenteral or oral administration to a patient in need thereof.

Mention of palonosetron is intended to include any of the alternative forms in which the palonosetron can be administered, such as salts, esters, hydrates, solvates, crystalline or amorphous polymorphs, racemic mixtures, enantiomeric isomers, etc. An example of a useful salt is the hydrochloride.

The following examples further describe certain specific aspects and embodiments of the invention and demonstrate the practice and advantages thereof. It is to be understood that the examples are given by way of illustration only and are not intended to limit the scope of the invention in any manner.

In the examples, impurity percentages are calculated based on the label drug content of a formulation.

### Example 1

### Formulation Comprising Palonosetron with EDTA

| Ingredient | mg/Vial |
|---|---|
| Palonosetron* | 0.25 |
| Mannitol | 207.5 |
| Edetate disodium dihydrate | 2.5 |
| Citric acid monohydrate | 18.5 |
| Trisodium citrate dihydrate | 7.8 |
| 1N NaOH and/or HCl | q.s. |
| Water‡ | q.s. |

*Provided as palonosetron hydrochloride.
‡Evaporates during the lyophilization process.

Manufacturing Process:

1. Dissolve mannitol, EDTA, citric acid monohydrate, and trisodium citrate dihydrate in water.

2. Dissolve palonosetron HCl in the solution.

3. Adjust the pH to 5±1 using sodium hydroxide or hydrochloric acid.

4. Make up the final volume with water and mix well.

5. Filter the solution through a 0.2 μm sterilization membrane filter.

6. Fill an aliquot of the solution into a glass vial and loosely cover with a slotted sterile bromobutyl rubber stopper.

7. Lyophilize the loosely covered vial in a freeze dryer.

8. After completion of lyophilization, stopper the vial completely by hydraulic pressing and seal the vial with a flip-off seal.

The lyophilized product is reconstituted using 5 mL of sterile water for injection prior to use.

A ready-to-use solution containing 0.05 mg/mL of palonosetron is also prepared using similar ingredients and a similar manufacturing process as above, except the lyophilization step is omitted.

The lyophilized product is stored for various time periods at various conditions, and samples are analyzed for pH upon reconstitution, and for drug and impurity contents. Results are in Table 1, where drug and impurity amounts are percentages of the label drug content.

TABLE 1

| | Storage Conditions | | | |
|---|---|---|---|---|
| | | | 40° C. and | 25° C. and |
| | | 60° C., | 75% RH, | 60% RH, |
| Parameter | Initial | 4 Weeks | 3 Months | 3 Months |
| pH | 5.08 | 5.08 | 5.01 | 4.99 |
| Drug Assay | 100.5 | 102.8 | 100.6 | 100.1 |

Drug-Related Impurities

| | | | | |
|---|---|---|---|---|
| Impurity A | ND | ND | ND | ND |
| Impurity B | 0.03 | 0.03 | 0.04 | 0.04 |
| Impurity C | 0.05 | 0.05 | 0.07 | 0.06 |
| Highest | 0.01 | 0.02 | ND | ND |
| Unidentified | | | | |
| Impurity | | | | |
| Total Impurities | 0.12 | 0.1 | 0.11 | 0.1 |

ND = Not detected.

### Example 2

### Formulation Comprising Palonosetron with EDTA

| Ingredient | mg/Vial |
|---|---|
| Palonosetron | 0.25 |
| Mannitol | 207.5 |
| Edetate disodium dihydrate | 2.5 |
| Citric acid monohydrate | 18.5 |
| Trisodium citrate dihydrate | 7.8 |
| 1N NaOH and/or HCl | q.s. |
| Water‡ | q.s. |

*Provided as palonosetron hydrochloride.
‡Evaporates during the lyophilization process.

Manufacturing process and reconstitution procedures for lyophilized formulation are similar to those described in Example 1, except that the pH of the product is adjusted to either 3±1 or 6±1.

A ready-to-use solution containing 0.05 mg/mL of palonosetron is also prepared using similar ingredients and a similar manufacturing process, except the lyophilization step is omitted.

### Example 3

### Formulation Comprising Palonosetron without EDTA

| Ingredient | mg/Vial |
|---|---|
| Palonosetron | 0.25 |
| Mannitol | 207.5 |
| Citric acid monohydrate | 18.5 |
| Trisodium citrate dihydrate | 7.8 |
| 1N NaOH and/or HCl | q.s. |
| Water‡ | q.s. |

*Provided as palonosetron hydrochloride.
‡ Evaporates during the lyophilization process.

Manufacturing process and reconstitution procedures are similar to those described in Example 1, except that the composition does not include edetate disodium dihydrate.

A ready-to-use solution containing 0.05 mg/mL of palonosetron is also prepared using similar ingredients and a similar manufacturing process, except the lyophilization step is omitted.

The lyophilized product is stored for various time periods at various conditions, and samples are analyzed for pH upon reconstitution, and for drug and impurity contents. Results are in Table 2, where drug and impurity amounts are percentages of the label drug content.

TABLE 2

| | Storage Conditions | | | |
|---|---|---|---|---|
| | | | 40° C. and | 25° C. and |
| | | 60° C. | 75% RH, | 60% RH, |
| Parameter | Initial | 4 Weeks | 3 Months | 3 Months |
| pH | 5.08 | 5.08 | 5.02 | 5.01 |
| Drug Assay | 102.2 | 102.6 | 98.5 | 98.5 |

Drug-Related Impurities

| | | | | |
|---|---|---|---|---|
| Impurity A | ND | ND | ND | ND |
| Impurity B | 0.03 | 0.05 | 0.04 | 0.03 |
| Impurity C | 0.05 | 0.05 | 0.03 | 0.05 |
| Highest | 0.01 | 0.04 | 0.04 | ND |
| Unidentified | | | | |
| Impurity | | | | |
| Total Impurities | 0.1 | 0.17 | 0.17 | 0.08 |

ND = Not detected.

### Example 4

### Formulation Comprising Palonosetron without EDTA

| Ingredient | mg/Vial |
|---|---|
| Palonosetron* | 0.25 |
| Mannitol | 207.5 |
| Trisodium citrate | 18.5 |
| Citric acid monohydrate | 7.8 |
| 1N NaOH and/or HCl | q.s. |
| Water‡ | q.s. |

*Provided as palonosetron hydrochloride.
‡Evaporates during the lyophilization process.

Manufacturing process and reconstitution procedures are similar to those described in Example 3, except that the pH of the product is adjusted to 3±1 or 6±1.

A ready-to-use solution containing 0.05 mg/mL of palonosetron is also prepared using similar ingredients and a similar manufacturing process, except the lyophilization step is omitted.

### Example 5

### Formulation Comprising Palonosetron with EDTA

| Ingredient | mg/Vial |
|---|---|
| Palonosetron* | 0.25 |
| Mannitol | 207.5 |
| EDTA | 2.5 |
| Sodium acetate | 15 |
| 1N NaOH and/or HCl | q.s. |
| Water‡ | q.s. |

*Provided as palonosetron hydrochloride.
‡Evaporates during the lyophilization process.

Manufacturing Process:

1. Dissolve EDTA, mannitol, and sodium acetate in water.

2. Dissolve palonosetron HCl in the solution.

3. Adjust the pH to 5±1 using sodium hydroxide or hydrochloric acid.

4. Dilute to the desired volume and mix well.

5. Filter the solution through a 0.2 μm sterilizing membrane filter.

6. Fill an aliquot of the filtrate into a glass vial and loosely cover with a slotted sterile bromobutyl rubber stopper.

7. Lyophilize the loosely covered vial in a freeze dryer.

8. After completion of lyophilization, stopper the vial completely by hydraulic pressing and seal the vial with a flip-off seal.

The lyophilized product is reconstituted using 5 mL of sterile water for injection prior to use.

A ready-to-use solution containing 0.05 mg/mL of palonosetron is also prepared using similar ingredients and a similar manufacturing process, except the lyophilization step is omitted.

### Example 6

### Formulation Comprising Palonosetron without EDTA

| Ingredient | mg/Vial |
|---|---|
| Palonosetron* | 0.25 |
| Mannitol | 207.5 |
| Sodium acetate | 15 |
| 1N NaOH and/or HCl | q.s. |
| Water‡ | q.s. |

*Provided as palonosetron hydrochloride.
‡Evaporates during the lyophilization process.

Manufacturing Process:

1. Dissolve mannitol and sodium acetate in water.

2. Dissolve palonosetron HCl in the solution.

3. Adjust the pH to 5±1 using sodium hydroxide or hydrochloric acid.

4. Dilute to the desired volume with water and mix well.

5. Filter the solution through a 0.2 μm sterilizing membrane filter.

6. Fill an aliquot of the solution into a glass vial and loosely cover with a slotted sterile bromobutyl rubber stopper.

7. Lyophilize the loosely covered vial in a freeze dryer.

8. After completion of lyophilization, stopper the vial completely by hydraulic pressing and seal the vial with a flip-off seal.

The lyophilized product is reconstituted using 5 mL of sterile water for injection, prior to use.

A ready-to-use solution containing 0.05 mg/mL of palonosetron is also prepared using similar ingredients and a similar manufacturing process, except the lyophilization step is omitted.

We claim:

1. A pharmaceutical formulation for parenteral administration, comprising: a) palonosetron or a pharmaceutically acceptable salt thereof; and b) a pharmaceutically acceptable carrier; wherein the formulation is substantially fee of chelating agents, antioxidants, or both.

2. The pharmaceutical formulation of claim 1 in liquid form, wherein palonosetron or a pharmaceutically acceptable salt thereof comprises palonosetron hydrochloride at a concentration about 0.05 mg/mL.

3. The pharmaceutical formulation of claim 1 in liquid form, having pH values about 3.5 to about 6.5.

4. The pharmaceutical formulation of claim 1, in a lyophilized form that can be reconstituted with an aqueous liquid.

5. The pharmaceutical formulation of claim 1, further comprising a pH adjusting agent.

6. The pharmaceutical formulation of claim 1, wherein a pharmaceutically acceptable carrier comprises at least one buffering agent and at least one tonicity contributor.

7. The pharmaceutical formulation of claim 6, wherein a buffering agent comprises an acetate, citrate, tartarate, phosphate, benzoate, or bicarbonate compound.

8. The pharmaceutical formulation of claim 6 in liquid form, wherein the formulation comprises about 10 to about 100 millimoles/mL of buffering agent.

9. The pharmaceutical formulation of claim 6, wherein a tonicity contributor comprises sodium chloride, potassium chloride, dextrose, mannitol, sorbitol, or lactose.

10. The pharmaceutical formulation of claim 6 in liquid form, wherein the formulation comprises about 10 mg/mL to about 80 mg/mL of a tonicity contributor.

11. The pharmaceutical formulation of claim 1, wherein a total palonosetron-related impurity content is less than about 1 percent by weight of a label palonosetron content.

12. A lyophilized solid containing a pharmaceutically acceptable salt of palonosetron, a tonicity contributor, and a buffering agent.

13. The pharmaceutical formulation of claim 12, being substantially free of chelating agents, antioxidants, or both.

14. The pharmaceutical formulation of claim 12, wherein a tonicity contributor comprises mannitol.

15. The pharmaceutical formulation of claim 12, wherein a buffering agent comprises a citrate salt.

16. The pharmaceutical formulation of claim 12, wherein a buffering agent comprises an acetate salt.

17. The pharmaceutical formulation of claim 12, which, after reconstitution with an aqueous liquid, comprises about 0.05 mg/mL palonosetron.

18. The pharmaceutical formulation of claim 12, which, after reconstitution with an aqueous liquid, has pH values about 3.5 to about 6.5.

19. The pharmaceutical formulation of claim 12, which, after reconstitution with an aqueous liquid, comprises about 10 to about 100 millimoles/mL of buffering agent.

20. The pharmaceutical formulation of claim 12, which, after reconstitution with an aqueous liquid, comprises about 10 mg/mL to about 80 mg/mL of a tonicity contributor.
